(19) United States
(12) Patent Application Publication
(10) Pub. No.: US 2022/0331181 A1
(43) Pub. Date: Oct. 20, 2022

(54) DIAGNOSTIC APPARATUS HAVING NEGATIVE PRESSURE ROOM

(71) Applicant: Jong Woon SONG, Gwangju (KR)

(72) Inventor: Jong Woon SONG, Gwangju (KR)

(21) Appl. No.: 17/418,073

(22) PCT Filed: Apr. 23, 2021
(86) PCT No.: PCT/KR2021/005210

§ 371 Date: Jun. 24, 2021

(30) Foreign Application Priority Data
May 18, 2020 (KR) 10-2020- 0058954

(51) Int. Cl. A61G 10/02; F24F 7/08; A61B 10/00; A61G 10/00

(52) U.S. Cl. CPC A61G 10/023; F24F 7/08; A61B 10/0051; A61G 10/005; F24F 2011/0005

(57) ABSTRACT

Proposed is a diagnostic apparatus including: an anteroom having a first door; a specimen obtainment room having a second door to be closed while the first door is opened after a person designated to be diagnosed enters the anteroom, and a specimen obtainment space where a specimen is obtained from the person; a diagnosis room being kept secluded from an atmosphere of each of the anteroom and the specimen obtainment room; and a negative pressure generator providing a negative pressure in such a manner that pressure in the anteroom is formed above pressure in the specimen obtainment room, wherein a specimen obtainment means for obtaining the specimen from the person in the specimen obtainment room is installed in a direction from the diagnosis room toward the specimen obtainment room, and the specimen obtainment means is sealed from the atmosphere of the specimen obtainment room.

### TECHNICAL FIELD

The present invention relates to a diagnostic apparatus having a negative pressure room.

### BACKGROUND ART

Usually, a specimen, such as sputum, is obtained from a patient through face-to-face contact between a medical team member and the patient. However, there is a concern that the medical team member or another patient may be infected while the specimen is obtained in a face-to-face contact manner.

As a technology of solving this problem, for example, an unmanned sputum collection booth for prevention of infection was disclosed in Korean Patent No. 10-1674482B1. In this booth, when a patient enters an anteroom, the entrance door of the anteroom is closed, and, at the same time, a door to a sputum collection room is opened. Thus, the booth is reliably sealed from the outside. In addition, the sputum collection room is maintained in a negatively pressurized state and thus is more reliably sealed from the outside. Thereby, infection of another patient or a test subject is suppressed while obtaining sputum from the patient or the test subject.

However, even in the booth in the related art, a doctor and the patient are still required to make a face-to-face contact with each other in the sputum collection room. Thus, although the sputum collection room is maintained in the negatively pressurized state, the doctor cannot be reliably prevented from being infected through the face-to-face contact with the patient.

In addition, in the booth in the related art, sputum obtainment tubes containing the collected sputum are stored together in the sputum collection room, to be discharged later out of the sputum collection room at once. For this reason, there occurs a problem in that the sputum obtainment tube is contaminated with microorganisms or infected with pathogens.
[Patent Document 1] Korean Patent No. 10-1674482B1

### Technical Problem

An objective of the present invention, which is made in view of this situation, is to suppress infection between a person designated to be diagnosed and a medical team member who obtains a specimen from the person designated to be diagnosed.

Another objective of the present invention is to maximally suppress infection between people who come in and go out successively, by automatically removing air particles in space around a moving path and automatically cleaning designated objects on the moving path in a case where specimens are successively obtained.

Still another objective of the present invention is to maximally reduce involvement of human work in obtaining specimens and thus maximally suppress infection in obtaining the specimens.

Still another objective of the present invention is to prevent a specimen collection tube from being contaminated with microorganisms.

Still another objective of the present invention is to provide a mobile diagnostic apparatus capable of being quickly and easily installed and operated in any place.

### Technical Solution

In order to achieve these objectives, according to an aspect of the present invention, there is provided a diagnostic apparatus including: an anteroom having a first door; a specimen obtainment room with a second door configured to maintain a closed state while the first door of the anteroom is opened after a person designated to be diagnosed enters the anteroom, and with a specimen obtainment space where a specimen is obtained from the person designated to be diagnosed; a diagnosis room to maintain a state of being secluded from both an atmosphere of the anteroom and an atmosphere of the specimen obtainment room; and a negative pressure generator providing a negative pressure to each of the anteroom and the specimen obtainment room in such a manner that the pressure in each of the anteroom and the specimen obtainment room is lower than the pressure in the diagnosis room, wherein a specimen obtainment means for obtaining the specimen from the person designated to be diagnosed present in the specimen obtainment room is installed in a direction from the diagnosis room toward the specimen obtainment room, and the specimen obtainment means is sealed from the atmosphere of the specimen obtainment room.

According to the present invention, the diagnosis room is secluded from the atmosphere of the anteroom and from the atmosphere of the specimen obtainment room. Thus, infection between the person designated to be diagnosed and a medical team member can be reliably suppressed. In addition, since the negative pressure is provided to each of the anteroom and the specimen obtainment room, pathogens can be reliably prevented from spreading from the specimen obtainment room to the anteroom and the outside.

The diagnostic apparatus may further include: a negatively pressurized room disinfection means for disinfecting the anteroom and the specimen obtainment room.

At this point, the negatively pressurized room disinfection means can be configured to provide disinfection while being moved upward and downward within the anteroom and the specimen obtainment room.

According to the present invention, since the anteroom and the specimen obtainment room can be regularly/irregularly disinfected using the negatively pressurized room disinfection means, infection between the persons designated to be diagnosed who enter and exit the anteroom and the specimen obtainment room can be more reliably prevented.

In the diagnostic apparatus, the diagnosis room may include a specimen collection tube handling compartment for providing a specimen collection tube to the specimen obtainment room as well as collecting the specimen collection tube from the specimen obtainment room.

In the diagnostic apparatus, a tube transportation path may be formed between the specimen obtainment room and the specimen collection tube handling compartment, the tube transportation path may include: a specimen collection tube reception compartment configured in such a manner that the specimen collection tube is transported from the specimen collection tube handling compartment toward the specimen obtainment room; and a specimen collection tube collection compartment configured in such a manner that the specimen collection tube is transported from the specimen obtainment room toward the specimen collection tube handling compartment.

In the diagnostic apparatus, the specimen collection tube reception compartment may be formed inclined from the specimen collection tube handling compartment toward the specimen obtainment room, and the specimen collection tube collection compartment may be formed inclined from the specimen obtainment room toward the specimen collection tube handling compartment.

According to the present invention, since the specimen collection tube can be safely moved between the specimen obtainment room and the diagnosis room, the pathogens can be reliably prevented from spreading. In addition, according to the present invention, since the specimen collection tube can be carried out of the diagnosis room whenever the medical team member finishes obtaining the specimen, other persons designated to be diagnosed in the diagnosis room can be further suppressed from being infected through the specimen collection tube.

In the diagnostic apparatus, the specimen collection tube handling compartment may include a tube transportation means for transporting the collected specimen collection tube to a predetermined place.

In the diagnostic apparatus, the tube transportation means may include: a tube accommodation unit for accommodating the specimen collection tube in an upright position; a transportation unit for transporting the tube accommodation unit along a transportation path; and a drive unit for driving the transportation unit.

In the diagnostic apparatus, the tube accommodation unit may include: a base portion; a carrying plate, installed on the base portion, for maintaining the specimen collection tube; and a vertical support portion extending vertically from the carrying plate in order to maintain the specimen collection tube in the upright position.

According to the present invention, the specimen collection tube being moved on the transportation unit is maintained in the upright position, and thus, damage to the specimen collection tube due to falling down of the specimen collection tube and the resulting spread of pathogens can be more reliably suppressed.

In the diagnostic apparatus, the specimen collection tube handling compartment may further include a tube disinfection means for disinfecting the specimen collection tube on the tube transportation means.

In the diagnostic apparatus, the tube transportation means may further include a horizontal rotation drive unit configured to transmit a rotational force in a horizontal direction to the tube accommodation unit, and the tube disinfection means may serve to disinfect the specimen collection tube accommodated in the tube accommodation unit rotated horizontally by the horizontal rotation drive unit.

According to the present invention, since the specimen collection tube is disinfected, the pathogens can be prevented from spreading from a surface of the specimen collection tube. Particularly, since the disinfection is provided while the specimen collection tube is rotated horizontally, the specimen collection tube can be more reliably disinfected.

In the diagnostic apparatus, the tube transportation means may further include a tube discharge means for discharging in a discharge direction the specimen collection tube reaching a downstream side of the transportation unit.

In the diagnostic apparatus, the tube discharge means may include a discharging direction rotation drive unit for rotating in the discharge direction the tube accommodation unit reaching the downstream side of the transportation unit, without deviating from the transportation unit.

According to the present invention, only the specimen collection tube accommodated in the tube accommodation unit is discharged without deviating the transportation unit, and thus the tube accommodation unit can be efficiently utilized.

In the diagnostic apparatus, the specimen obtainment means may include: an arm entry portion; an arm contact portion extending from the arm entry portion toward the specimen obtainment room; and a cover portion configured to open and close the arm entry portion.

In the diagnostic apparatus, the specimen obtainment means may further include a pressure detection means for detecting pressure within the arm body portion in a state where the cover portion is closed.

In the diagnostic apparatus, the pressure detection means may include: a through-hole for measuring pressure, the through-hole being formed in the cover portion; an air hose being connected to the through-hole and extending to the outside; and a pressure measurement means connected to the air hose.

According to the present invention, since abnormal pressure within the specimen obtainment means can be detected, spread of the pathogens between the diagnosis and the specimen obtainment room can be recognized in advance and thus can be prevented.

In the diagnostic apparatus, the specimen obtainment means may further include a heater configured to heat the arm body portion.

According to the present invention, the inside of the specimen obtainment means can maintain a clean state by heating the specimen obtainment means.

The diagnostic apparatus may further include an energy storage system or an uninterruptible power supply.

According to the present invention, electric power can be stably provided to the diagnostic apparatus.

The diagnostic apparatus may be a mobile diagnostic apparatus.

According to the present invention, the mobile diagnostic apparatus capable of being easily installed and operated in a short time in any place can be provided.

### Advantageous Effects

According to the present invention, infection between a person designated to be diagnosed and a medical team member who obtains a specimen from the person designated to be diagnosed can be suppressed.

In addition, according to the present invention, infection between people who come in and go out successively by automatically removing air particles in space around a moving path and automatically cleaning designated objects on the moving path can be suppressed to a maximum in a case where specimens are successively obtained.

In addition, according to the present invention, involvement of human work in obtaining specimens can be suppressed to a minimum, and thus infection in obtaining the specimens can be suppressed to a maximum.

In addition, according to the present invention, a specimen collection tube can be prevented from being contaminated with microorganisms.

In addition, a mobile diagnostic apparatus capable of being easily installed and operated in a short time in any place can be provided.

### DESCRIPTION OF DRAWINGS

FIG. 1 is a plane view illustrating a diagnostic apparatus according to an embodiment of the present invention;

FIG. 2 is a plane view illustrating a negatively pressurized room disinfection means 50 of the diagnostic apparatus according to the embodiment of the present invention;

FIG. 3 is a perspective view illustrating a tube transportation path according to the embodiment of the present invention;

FIG. 4 is a perspective view illustrating a specimen obtainment means according to the embodiment of the present invention;

FIG. 5 is a perspective view illustrating a tube transportation means according to the embodiment of the present invention;

FIG. 6 is an enlarged view illustrating the downstream side A of the tube transportation means according to the embodiment of the present invention;

FIG. 7 is a view illustrating that according to the embodiment of the present invention, a specimen collection tube is discharged from the tube transportation means;

FIG. 8 is a view illustrating a configuration of an entire system according to the embodiment of the present invention; and

FIG. 9 is a flowchart for a specimen obtainment method using the diagnostic apparatus according to the embodiment of the present invention.

### MODE FOR INVENTION

A diagnostic apparatus according to an embodiment of the present invention will be described below with reference to the accompanying drawings.

The terms “secluded”, “sealed”, and the like throughout the present specification refer to a state where an atmosphere is prevented from moving between objects designated to be secluded or sealed. For example, as described below, seclusion of a diagnosis room from an anteroom and a specimen obtainment room means that pathogens cannot be spread between the diagnosis room and the anteroom/specimen obtainment room.

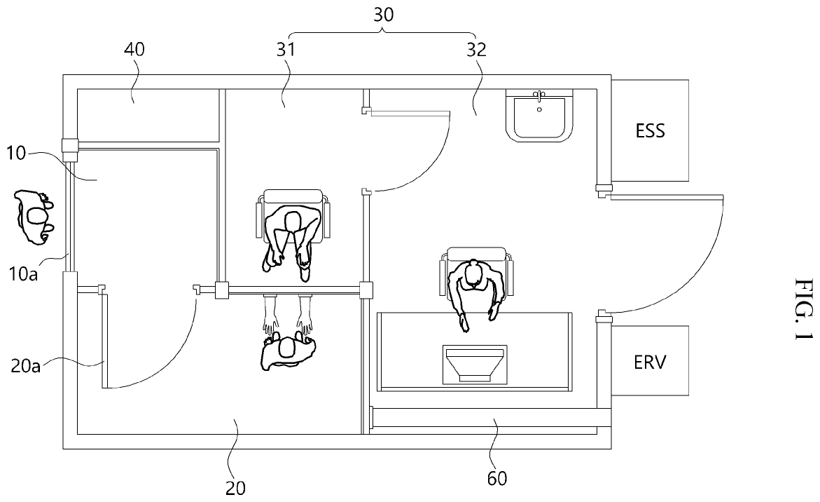

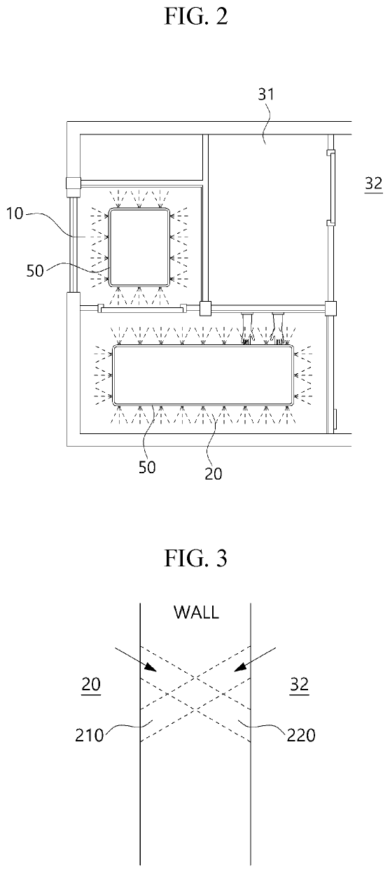

FIGS. 1 and 2 illustrate a diagnostic apparatus 1 according to an embodiment of the present invention.

The diagnostic apparatus 1 according to the embodiment of the present invention may be configured, for example using a mobile container house as disclosed in Korean Patent No. 10-1401209 granted to the inventor of the present invention. In this case, the diagnostic apparatus 1 is a mobile diagnostic apparatus.

The diagnostic apparatus 1 according to the present invention always needs to be supplied with electric power. Therefore, it is desirable that the diagnostic apparatus 1 includes an energy storage system (ESS) or an uninterruptible power supply (UPS). The ESS or the UPS, as illustrated, may be installed on a predetermined portion of the diagnostic apparatus 1.

The diagnostic apparatus 1 according to the embodiment of the present invention includes an anteroom 10, a specimen obtainment room 20, a diagnosis room 30, and a negative pressure generation room 40. A person, such as a patient, who is designated to be diagnosed, comes in and goes out through the anteroom 10. The specimen obtainment room 20, adjacent to the anteroom 10, accommodates persons designated to be diagnosed who come in through the anteroom 10. A specimen is obtained from the person designated to be diagnosed in the specimen obtainment room 20. The diagnosis room 30, secluded from the specimen obtainment room 20, accommodates a medical team member that obtains a specimen from the person designated to be diagnosed present in the specimen obtainment room 20. The negative pressure generation room 40 serves to generate a negative pressure and maintaining the anteroom 10 and the specimen obtainment room 20 in a negatively pressurized state. In addition, the diagnostic apparatus 1 may further include a negatively pressurized room disinfection means 50. The negatively pressurized room disinfection means 50 serves to disinfect the anteroom 10 and the specimen obtainment room 20 which maintain the negatively pressurized state.

The diagnosis room 30 is divided into a diagnosis compartment 31 and a specimen collection tube handling compartment 32. The diagnosis compartment 31 serves to accommodate a medical team member that obtains the specimen from the person designated to be diagnosed and the like. The specimen collection tube handling compartment 32 serves to provide and receive a specimen collection tube to and from the person designated to be diagnosed.

A door 10a, such as a sliding door, is provided to the anteroom 10. When the person designated to be diagnosed approaches the anteroom 10 according to an announcement or the like, the door 10a is automatically opened. After the person designated to be diagnosed enters the anteroom 10, when the door 10a to the anteroom 10 is completely closed, a door 20a (for example, a swing door) to the specimen obtainment room 20 is opened. Through the door 20a, the person designated to be diagnosed enters a specimen obtainment space in the specimen obtainment room 20.

The specimen obtainment room 20 may include a non-contact thermometer used by the medical team member within the diagnosis compartment 31. The non-contact thermometer serves to measure a body temperature of the person designated to be diagnosed without face-to-face contact with the person designated to be diagnosed. The non-contact thermometer, for example, may be installed on a wall of the specimen obtainment room 20 adjoining the diagnosis compartment 31. A button for operating the non-contact thermometer is provided on the diagnosis compartment 31 side. The medical team member can operate the button and thus can measure the body temperature of the person designated to be diagnosed. In addition, a means necessary for diagnosis and physical examination of the person designated to be diagnosed may be provided in the specimen obtainment room 20.

For example, specimen obtainment gloves 300, as a specimen obtainment means passing through the wall of the diagnosis room 30 and extending forward, are provided in the specimen obtainment room 20. When using the specimen obtainment gloves 300, the medical team member within the diagnosis compartment 31 puts his/her arms into the specimen obtainment gloves 300, respectively, and obtains a specimen from the person designated to be diagnosed. The specimen obtainment gloves 300 is sealed from an atmosphere of the specimen obtainment room 20.

In addition, cover portions capable of opening and closing the specimen obtainment gloves 300 may be formed on a wall surface, on the specimen obtainment gloves 300 side, of the diagnosis compartment 31. When the medical team member stop obtaining the specimen to take a rest or the like, the specimen obtainment gloves 300 are closed with the cover portions, respectively. Thus, pathogens can be more reliably prevented from spreading from the specimen obtainment room 20 into the diagnosis compartment 31. A more specific configuration of the specimen obtainment glove 300 will be described below.

In addition, in the specimen obtainment room 20, there may be provided an information input means through which the person designated to be diagnosed can input information related to him/her. When various pieces of information (personal information, symptom information related to infection or disease, and the like) on the person designated to be diagnosed are input, the various pieces of information can be transferred to a server in a remote hospital, a disease control and prevention center, or the like. In addition, among the input pieces of information, pieces of information (for example, pieces of information for identifying the person designated to be diagnosed, such as a name and a resident registration number) required to be attached on the specimen collection tube may be printed through an output device such as a printer. For example, the information input means, such as a touch screen, may be provided on a wall surface of the specimen obtainment room 20. The output device such as the printer is provided, for example, in the specimen collection tube handling compartment 32.

The specimen obtainment room 20, as illustrated in FIG. 3, may include a specimen collection tube reception compartment 210 and a specimen collection tube collection compartment 220. The specimen collection tube reception compartment 210 serves to receive the specimen collection tube from the specimen collection tube handling compartment 32. The specimen collection tube collection compartment 220 serves to collect the specimen collection tube containing the specimen from the person designated to be diagnosed. The specimen collection tube reception compartment 210 and the specimen collection tube collection compartment 220, for example, may be provided on a wall between the specimen obtainment room 20 and the specimen collection tube handling compartment 32. The specimen collection tube reception compartment 210 and the specimen collection tube collection compartment 220 correspond to a tube transportation path according to the present invention.

The specimen collection tube reception compartment 210 and the specimen collection tube collection compartment 220 may be formed as the tube transportation path formed obliquely in a wall between the specimen collection tube handling compartment 32 and the specimen obtainment room 20 in order to safely receive and return the specimen collection tube without any damage through the wall therebetween. The specimen collection tube is transported along the tube transportation in the direction of an arrow in FIG. 3.

The medical team member outputs the information obtained from the person designated to be diagnosed through the printer present in the specimen collection tube handling compartment 32, attaches the output information on the specimen collection tube, and then provides the specimen collection tube, on which the information is attached, to the person designated to be diagnosed present in the specimen obtainment room 20 through the specimen collection tube reception compartment 210. The medical team member present in the diagnosis compartment 31 obtains a specimen from the person designated to be diagnosed, places the obtained specimen into the specimen collection tube and delivers the specimen collection tube containing the specimen to the person designated to be diagnosed. The person designated to be diagnosed returns the specimen obtainment containing the specimen tube to the specimen collection tube handling compartment 32 through the specimen collection tube collection compartment 220.

Shutters (refer to FIG. 5) are provided on openings, respectively, on both sides of the specimen collection tube reception compartment 210. Shutters (refer to FIG. 5) are also provided on openings, respectively, on both sides of the specimen collection tube collection compartment 220. When one shutter on one side of the specimen collection tube reception compartment 210 is opened, the shutter on the other side is required to be closed in order to maintain a state where the specimen obtainment room 20 and the specimen collection tube handling compartment 32 are secluded or sealed from each other. The same is true for the specimen collection tube collection compartment 220. That is, both shutters are not required to be opened at the same time.

According to the present embodiment, the shutter is a type of slide built into the wall. However, instead of the shutter. A cover that is to be attached on each of the openings in the specimen collection tube reception compartment 210 and the specimen collection tube collection compartment 220 may be provided. In order to maintain the sealed state more reliably, a damper preventing air from flowing backward may be additionally installed in an internal path in each of the specimen collection tube reception compartment 210 and the specimen collection tube collection compartment 220.

In addition, other means (for example, a disinfected cotton swab and the like) necessary to obtain a specimen are provided in the specimen obtainment room 20. Among these means, the person designated to be diagnosed may provide a necessary means to the medical team member present in the diagnosis compartment 31 along with the specimen collection tube. Alternatively, the medical team member present in the specimen collection tube handling compartment 32 may transfer another means necessary to obtain a specimen to the person designated to be diagnosed present in the specimen obtainment room 20. In this manner, other means necessary to obtain a specimen may be prevented more reliably from being contaminated with microorganisms.

The specimen obtainment room 20 and the diagnosis compartment 31 are secluded from each other, and the inside of the specimen obtainment means (for example, the specimen obtainment gloves) is also sealed from the atmosphere of the specimen obtainment room 20. Therefore, the medical team member can safely obtain a specimen from the person designated to be diagnosed through the specimen obtainment means (for example, the specimen attainment gloves). In addition, while the medical team member takes a rest or the like, the specimen obtainment means is closed. Thus, the protection of the medical team member against infection can be enhanced.

The diagnosis compartment 31 accommodates the medical team member who obtains the specimen from the person designated to be diagnosed. The specimen collection tube handling compartment 32 accommodates the medical team member and functions as a space for providing and collecting the specimen collection tube.

In the specimen collection tube handling compartment 32, there is provided an output means, such as a printer, for outputting information to be attached on the specimen collection tube among pieces of information input through the information input means provided in the specimen obtainment room 20. In the specimen collection tube handling compartment 32, in order to store the collected specimen collection tube in a separate place, there is provided a tube transportation means for transporting the specimen collection tube. The tube transportation means will be described in more detail below.

In the negative pressure generation room 40, there is provided a negative pressure generator for maintaining the anteroom 10 and the specimen obtainment room 20 in the negatively pressurized state. In some cases, a negative pressure may be generated only for the specimen obtainment room 20. In addition, a position of the negative pressure generation room 40 is not limited to a position illustrated in FIG. 1. The negative pressure generation room 40 may be positioned in any position where the negative pressure can be generated for the anteroom 10 and the specimen obtainment room 20.

In addition, according to the present embodiment, the negative pressure generator is provided in the negative pressure generation room 40. However, instead of in a specific space within the apparatus, the negative pressure generator may be provided in a state of being attached to the outside (for example, a ceiling or an external wall) of the diagnostic apparatus 1.

The negative pressure generator may be realized, for example, as a ventilation system that draws out an amount of air that is larger than an amount of air introduced indoors.

The anteroom 10 and the specimen obtainment room 20 are maintained in the negatively pressurized state, and thus the diagnosis room 30 is relatively maintained in a positive pressure state. In addition, a positive pressure generator (for example, an energy recovery ventilator (ERV)) for actively providing a positive pressure to the diagnosis room 30 may be installed separately as illustrated. A position in which the positive pressure generator is installed is not limited to a position illustrated. The positive pressure generator may be positioned at a different position, such as a ceiling of the apparatus, where the positive pressure can be efficiently provided to the diagnosis room 30. In a case where the energy recovery ventilator is used as the positive pressure generator, the inside of the diagnosis room 30 can be maintained in the positive pressure state by using an external air suction function of the energy recovery ventilator.

In a general ward equipped with the negative pressure generator, a pressure difference of approximately 2.5 Pa between a negatively pressurized room and the outside is maintained. According to the present embodiment, a pressure difference of up to approximately 5 Pa can be maintained by utilizing the positive pressure generator.

In this manner, a pressure difference between the diagnosis room 30 and each of the anteroom 10 and the specimen obtainment room 20 can be further increased by actively generating the positive pressure for the diagnosis room 30. Thus, the state where the diagnosis room 30 and each of the anteroom 10 the specimen obtainment room 20 are secluded from each other can be maintained more reliably. Therefore, the infection between the person designated to be diagnosed and the medical team member can be prevented more reliably.

In addition, the anteroom 10 is frequently brought into contact with the atmosphere through the door 10a, and the door 20a to the specimen obtainment room 20 are always closed while the door 10a to the anteroom 10 is opened. Thus, the pressure in the specimen obtainment room 20 is always maintained below the pressure in the anteroom 10. Therefore, pathogens can be reliably prevented from spreading from the specimen obtainment room 20 to the anteroom 10 and the outside.

The negatively pressurized room disinfection means 50 is a means for regularly/irregularly disinfecting the anteroom 10 and the specimen obtainment room 20 that need to maintain the negatively pressurized state. As illustrated in FIG. 2, for example, the negatively pressurized room disinfection means 50 installed on a ceiling portion of each of the anteroom 10 and the specimen obtainment room 20 disinfects a wall surface and a bottom surface of each of the anteroom 10 and the specimen obtainment room 20 while repeatedly ascending and descending.

Three types of nozzles may be provided on the negatively pressurized room disinfection means 50. A first type of nozzle is a nozzle for discharging a disinfecting solution, a second type of nozzle is a nozzle for discharging a hot cleaning solution (for example, hot water), and a third type of nozzle is a nozzle for discharging a cold cleaning solution. The medical team member present in the diagnosis room 30 can operate the negatively pressurized room disinfection means 50 by pushing a button therefore provided in the diagnosis room 30.

For example, by the medical team member present in the diagnosis compartment 31 pushing the button provided therein, the disinfecting solution, the hot cleaning solution, and the cold cleaning solution can be discharged in this order through the first type of nozzle, the second type of nozzle, and the third type of nozzle, respectively.

First and second nozzles for disinfecting the specimen obtainment gloves 300 may be provided to the negatively pressurized room disinfection means 50 in such a manner as to face the specimen obtainment gloves 300. The medical team member present in the diagnosis compartment 31 discharges the disinfection solution and the hot cleaning solution in this order through the first nozzle and the second nozzle, respectively, by pushing the button provided in the diagnosis compartment 31.

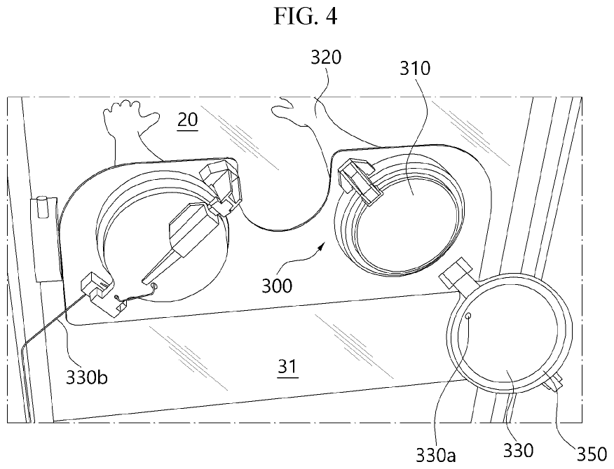

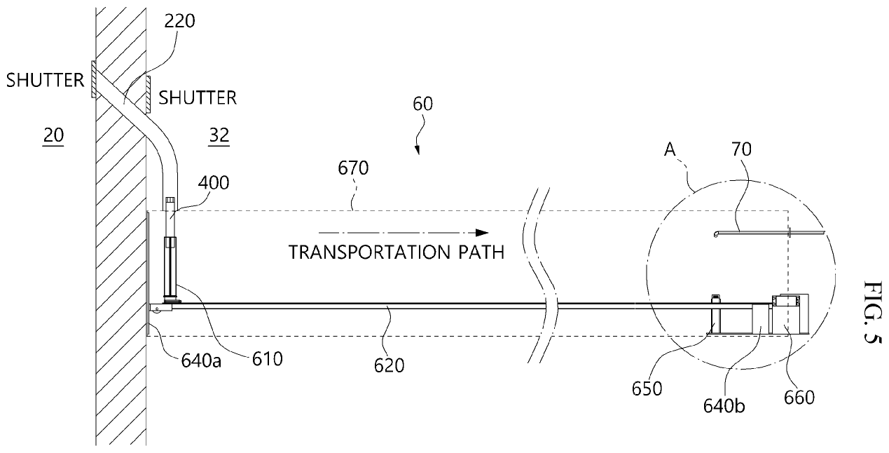

FIG. 4 illustrates a detailed configuration of each of the specimen obtainment gloves 300 protruding from the diagnosis compartment 31 toward the specimen obtainment room 20. The specimen obtainment gloves 300 includes an arm entry portion 310, an arm body portion 320, and a cover portion 330.

The arm entry portion 310 may be made of rubber packing or the like. The arm body portion 320 may be made of elastic material, such as rubber. However, if it can be assured that the diagnosis compartment 31 and the specimen obtainment room 20 are sealed from each other, the arm entry portion 310 and the arm body portion 320 may be made of any material.

The arm entry portion 310 and the arm body portion 320 are sealed from the specimen obtainment room 20. Therefore, the medical team member within the diagnosis compartment 31 can be prevented from being infected with pathogens within the specimen obtainment room 20. The medical team member within the diagnosis compartment 31 can insert his/her arms into the arm body portions 320 through the arm entry portions 310, respectively, and then can obtain the specimen from the person designated to be diagnosed.

The cover portion 330 serves to open or close the arm entry portion 310. While the specimen is obtained, the cover portion 330 may be in an open state. While the specimen is not obtained, the cover portion 330 may be in a closed state where the arm entry portion 310 is enclosed.

In addition, a through-hole 330a for measuring pressure is formed in the cover portion 330. An air hose 330b connected to a solenoid valve box (not illustrated) including a manometer, a solenoid valve, and the like is connected to an opening 330a for measuring pressure. With operation of the solenoid valve box, pressure within the arm body portion 320 can be measured through the air hose 330b and the through-hole 330a for measuring pressure.

In a normal situation, the measured pressure is maintained in a predetermined normal range. However, when an abnormal situation occurs such as damage to a part of the arm connection portion 320, the measured pressure will fall out of a normal range. When it is detected that the measured pressure falls out of the normal range, an alert to an emergent situation is caused through an alarming means, such as a speaker.

The solenoid valve box constitutes a pressure detection means according to the present invention. The solenoid valve box, the air hose 330b, the through-hole 330a for measuring pressure constitute a pressure detection means according to the present invention.

A heater 350 may be mounted on the cover portion 330, to heat the inside of the arm body portion 320 for sterilization when closed.

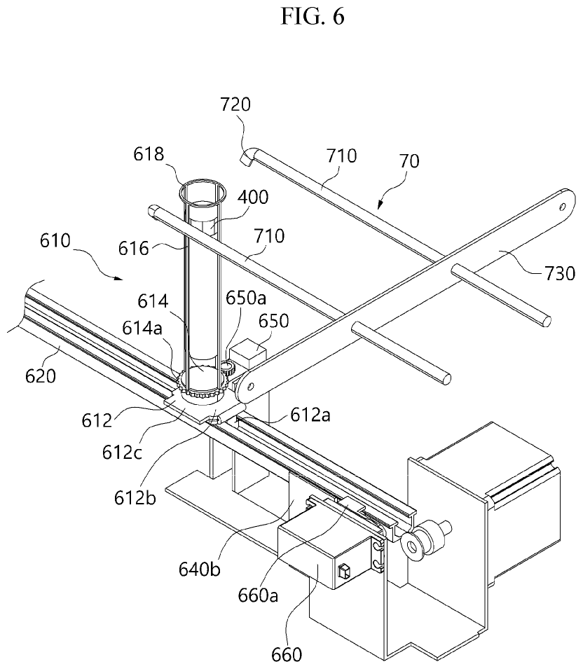

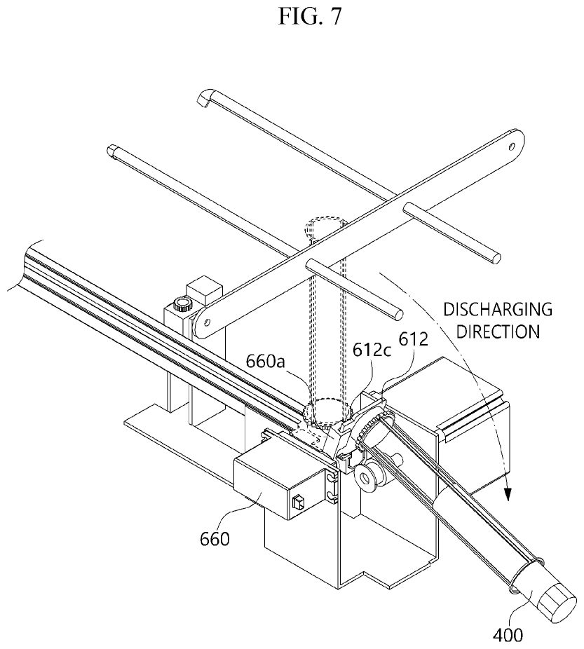

FIGS. 5 to 7 illustrate a specific configuration of a tube transportation means 60 provided in the specimen collection tube handling compartment 32. FIG. 5 illustrates an entire configuration of the tube transportation means 60. FIG. 6 is an enlarged view illustrating the downstream side (a portion indicated by a closed curved line A in FIG. 5) of the tube transportation means 60. FIG. 7 is a view illustrating that a specimen collection tube 400 is discharged.

The specimen collection tube handling compartment 32 includes the tube transportation means 60 for transporting along a transportation path the specimen collection tube 400 returned to the specimen collection tube collection unit 220 provided between the specimen collection tube handling compartment 32 and the specimen obtainment room 20.

The tube transportation means 60 includes a tube accommodation unit 610, a transportation unit 620, and a transportation drive unit 630. The tube accommodation unit 610 serves to accommodate in the upright position the specimen collection tube 400 placed into the specimen collection tube collection compartment 220. The transportation unit 620 serves to transport the tube accommodation unit 610 in a maintained state along the transportation path. The transportation drive unit 630 serves to drive the transportation unit 620. The transportation unit 620 and the transportation drive unit 630 may be realized, for example, as a linear motor drive system.

A tube disinfection means 70 is provided on the downstream side of the tube transportation means 60. The tube disinfection means 70 serves to disinfect the specimen collection tube 400 accommodated in the tube accommodation unit 610.

The tube transportation means 60 may further include transportation support means 640a and 640b. The transportation support means 640a and 640b serve for supporting the transportation unit 620. The transportation support means 640a may be mounted on a wall, on the specimen obtainment room 20 side, of the specimen collection tube handling compartment 32, and the transportation support means 640b may be mounted on the ground.

The tube transportation means 60 may further include a horizontal rotation drive unit 650 and a discharging direction rotation drive unit 660. The horizontal rotation drive unit 650 transfers a rotational force in the horizontal direction to the tube accommodation unit 610. The discharging direction rotation drive unit 660 serves to rotating the tube accommodation unit 610 in a discharge direction (FIG. 7). A housing 670 may be further provided. The housing 670 serves to accommodate one or several constituent elements of or all constituent elements of the tube transportation means 60.

The tube accommodation unit 610 includes a base portion 612, a carrying plate 614, and a vertical support portion 616. The base portion 612 is engaged with the transportation unit 620 and is moved along with the transportation unit 620. The carrying plate 614 is rotatably installed on the base portion 612 and serves to carry the specimen collection tube 400. The vertical support portion 616 extends vertically above the carrying plate 614. In addition, a ring-shaped connection portion 618 may be further included. The ring-shaped connection portion 618 is connected to the top portion of the vertical support unit 616.

The base portion 612 is configured to include an engagement portion 612a and a base plate 612b. The engagement portion 612a is engaged with the transportation unit 620 from below. The base plate 612b is hinge-combined with the engagement portion 612a to provide rotation of the engagement portion 612a toward the downstream side of the transportation unit 620. A protrusion piece 612c protruding toward a flank surface of the transportation unit 620 is installed on the base plate 612b.

The protrusion piece 612c is engaged with a protrusion piece accommodation portion 660a attached to the discharging direction rotation drive unit 660 and thus is rotated in the discharge direction (FIG. 7) by a rotational power transferred from the discharging direction rotation drive unit 660. The specimen collection tube 400 accommodated in the tube accommodation unit 610 is accordingly discharged out of the tube transportation means 60. The discharging direction rotation drive unit 660 constitutes a tube discharge means according to the present invention.

According to the present embodiment, four vertical support units 616 are formed in the shape of a support pin, but is not limited to the number thereof. The vertical support units 616 capable of maintaining the specimen collection tube 400 in the upright position without the specimen collection tube 400 falling down on the carrying plate 614 are not limited in number. The ring-shaped connection portion 618 has a ring-shaped opening through which the specimen collection tube 400 is reliably introduced into the tube accommodation unit 610 and serves to keep the vertical support unit 616 unshaken in such a manner that the vertical support unit 616 more reliably maintains the specimen collection tube 400 in the upright position.

A serrated wheel portion 614a is formed on a circumferential surface of the carrying plate 614. The serrated wheel portion 614a is engaged with a serrated wheel portion 650a of the horizontal rotation drive unit 650. When the carrying plate 614 is transported from the upstream side of the tube accommodation unit 610 and reaches the horizontal rotation drive unit 650, the serrated wheel portions 614a and 650a are engaged with each other. In this state, the horizontal rotation drive unit 650 transfers a motive power to the serrated wheel portion 650a, and the carrying plate 614 is accordingly rotated in the horizontal direction.

The tube disinfection means 70 sprays the disinfecting solution to the carrying plate 614 in rotation. The tube disinfection means 70 includes a disinfecting solution pipe 710, a nozzle portion 720, and a pipe support portion 730. The disinfecting solution pipe 710 serves to transport the disinfecting solution from a disinfecting solution supply source (not illustrated). The nozzle portion 720 serves to spray the disinfecting solution transported through the disinfecting solution pipe 710. The pipe support portion 730 serves to support the disinfecting solution pipe 710.

The disinfected specimen collection tube 400 is moved to the discharging direction rotation drive unit 660 in a state of being positioned in the tube accommodation unit 610. The protrusion piece accommodation portion 660a is attached to the discharging direction rotation drive unit 660. The protrusion piece accommodation portion 660a serves to accommodate in an engaged manner the protrusion piece 612c formed on the base portion 614. The discharging direction rotation drive unit 660 drives the protrusion piece accommodation portion 660a in a rotational direction in a state where the protrusion piece 612c formed on the base portion 612 is accommodated in the protrusion piece accommodation portion 660a in an engaged manner. Then, the tube accommodation unit 610, as illustrated, is rotated in the vertical downward direction and discharges the specimen collection tube 400 therein to the outside.

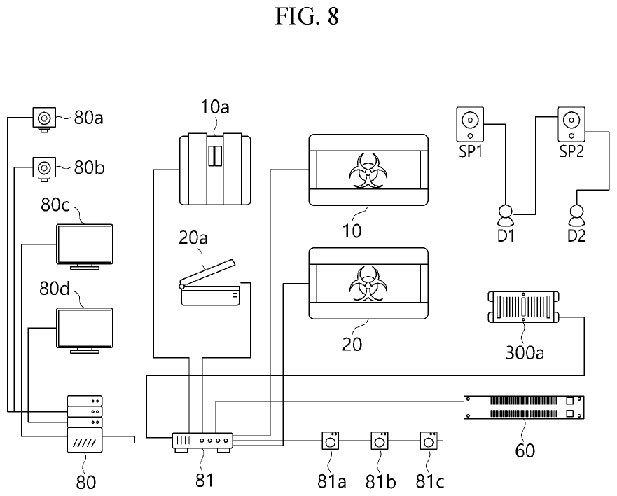

FIG. 8 illustrates a control system for controlling the entire operation of the diagnostic apparatus 1 according to the embodiment of the present invention.

An integrated controller 80 controls operation of a camera 80a for the door 10a to the anteroom 10, operation of a camera 80b for the specimen obtainment room 20, operation of a computer 80c used by the medical team member in the diagnosis room 30, operation of a computer 80d used by the person designated to be diagnosed in the specimen obtainment room 20, and operation of an input and output controller 81.

The input and output controller 81 is responsible for control that opens and closes the door (sliding door) 10a to the anteroom 10 and the door (swing door) 20a to the specimen obtainment room 20, control of operations, such as an operation of disinfecting the anteroom 10 and the specimen obtainment room 20, control of a module 300a managing the specimen obtainment gloves 300, control of operation of the tube transportation means 60, and control operation of various sensors. The various sensors include a temperature/humidity/pressure sensor 81a for the surrounding atmosphere, a temperature/humidity/pressure sensor 81b for the specimen obtainment room 20, a temperature/humidity/pressure sensor 81c for the diagnosis room 30, and a manometer 360 for the specimen obtainment means.

A microphone used by a medical team member D1 to page the person designated to be diagnosed and provide information and guidance to the person designated to be diagnosed is connected to speakers installed in the diagnostic apparatus 1, for example, a speaker SP1 installed on the door 10a to the anteroom 10 and a speaker S2 installed in the specimen obtainment room 20. A microphone used by a medical team member D2 who performs medical treatment in person, such as obtaining the specimen from the person designated to be diagnosed is also connected to the speaker SP2 in the specimen obtainment room 20 to communicate with the person designated to be diagnosed within the specimen obtainment room 20.

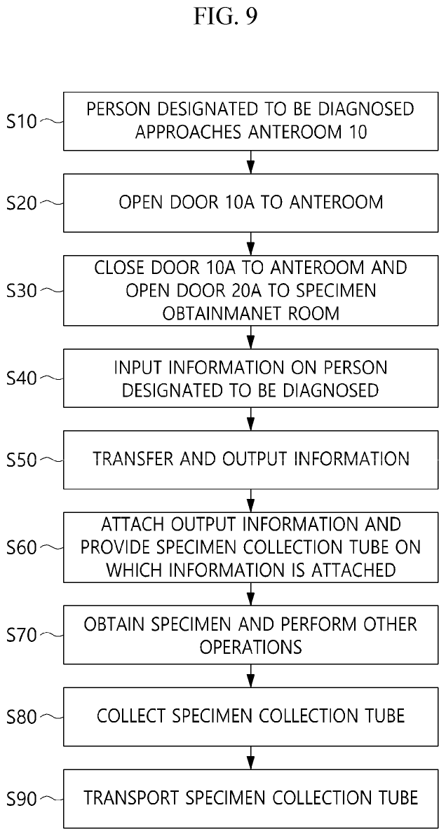

A specimen obtainment method using the diagnostic apparatus 1 according to the present invention will be described below with reference to the flowchart in FIG. 9.

First, when the person designated to be diagnosed is paged through the speaker or the like, the person designated to be diagnosed approaches the anteroom 10 (S10). When the person designated to be diagnosed is present in a predetermined range of distance from the anteroom 10, the door 10a to the anteroom 10 is opened (S20).

After the person designated to be diagnosed enters the anteroom 10, the door 10a to the anteroom 10 is closed, and, at the same time, the door 20a to the specimen obtainment room 20 is opened (S30).

When the person designated to be diagnosed enters the specimen obtainment room 20, the person designated to be diagnosed inputs information related to him/her in person through the information input means provided on the wall surface of the specimen obtainment room 20 (S40). At this time, through the speaker or the information input means, an instruction to input information may be provided, and/or an information input method or the like may be provided.

The input information may be transferred to an external designated server (in a hospital, a disease control and prevention center, or the like), and the information required to be attached on the specimen collection tube is output separately through a printer or the like (S50).

The medical team member within the specimen collection tube handling compartment 32 attaches the output information on the specimen collection tube and provides the specimen collection tube, on which the information is attached, to the person designated to be diagnosed (S60). At this time, the specimen collection tube is provided to the person designated to be diagnosed through the specimen collection tube reception compartment 210 provided on the wall of the specimen obtainment room 20.

The person designated to be diagnosed transfers the received specimen collection tube and another means (for example, a disinfected cotton swab or the like) to the medical team member present in the diagnosis compartment 31 through the specimen obtainment gloves 300. The medical team member obtains the specimen from the person designated to be diagnosed using the transferred specimen collection tube and other means and then transfers the specimen collection tube containing the specimen to the person designated to be diagnosed (S70).

The person designated to be diagnosed who receives the specimen collection tube containing the specimen places the specimen collection tube containing the specimen into the specimen collection tube collection compartment 220 provided in the specimen collection tube handling compartment 32 (S80).

The collected specimen collection tube is discharged into the specimen collection tube handling compartment 32 or a predetermined external place, through the tube transportation means 60 provided in the specimen collection tube handling compartment 32 (S90).

The preferred embodiment of the present invention is described above, and the present invention is not limited to the preferred embodiment. Any modification example that falls within the technical scope of the present invention falls within the scope of claim defining the present invention.

1. A diagnostic apparatus comprising:
an anteroom having a first door;
a specimen obtainment room having a second door configured to maintain a closed state while the first door of the anteroom is opened after a person designated to be diagnosed enters the anteroom, and a specimen obtainment space where a specimen is obtained from the person designated to be diagnosed;
a diagnosis room configured to maintain a state of being secluded from both an atmosphere of the anteroom and an atmosphere of the specimen obtainment room; and
a negative pressure generator providing a negative pressure to the anteroom and the specimen obtainment room in such a manner that each of the pressure in the anteroom and the specimen obtainment room is lower than the pressure in the diagnosis room,
wherein a specimen collection means for obtaining the specimen from the person designated to be diagnosed present in the specimen obtainment room is installed in a direction from the diagnosis room toward the specimen obtainment room, and the specimen collection means is sealed from the atmosphere of the specimen obtainment room.

2. The diagnostic apparatus of claim 1, further comprising:
a negatively pressurized room disinfection means for disinfecting the anteroom and the specimen obtainment room.

3. The diagnostic apparatus of claim 2, wherein the negatively pressurized room disinfection means provides disinfection while being moved upward and downward within the anteroom and within the specimen obtainment room.

4. The diagnostic apparatus of claim 1, wherein the diagnosis room comprises:
a specimen collection tube handling compartment for providing a specimen collection tube to the specimen obtainment room and collecting the specimen collection tube from the specimen obtainment room.

5. The diagnostic apparatus of claim 4, wherein a tube transportation path is formed between the specimen obtainment room and the specimen collection tube handling compartment,
wherein the tube transportation path comprises:
a specimen collection tube reception compartment configured in such a manner that the specimen collection tube is transported from the specimen collection tube handling compartment toward the specimen obtainment room; and
a specimen collection tube collection compartment configured in such a manner that the specimen collection tube is transported from the specimen obtainment room toward the specimen collection tube handling compartment.

6. The diagnostic apparatus of claim 5, wherein the specimen collection tube reception compartment is formed inclined from the specimen collection tube handling compartment toward the specimen obtainment room, and the specimen collection tube collection compartment is formed inclined from the specimen obtainment room toward the specimen collection tube handling compartment.

7. The diagnostic apparatus of claim 4, wherein the specimen collection tube handling compartment comprises:
a tube transportation means for transporting the collected specimen collection tube to a predetermined place.

8. The diagnostic apparatus of claim 7, wherein the tube transportation means comprises:
a tube accommodation unit for accommodating the specimen collection tube in an upright position;
a transportation unit for transporting the tube accommodation unit along a transportation path; and
a drive unit for driving the transportation unit.

9. The diagnostic apparatus of claim 8, wherein the tube accommodation unit comprises:
a base portion;
a carrying plate, installed on the base portion, for maintaining the specimen collection tube; and
a vertical support portion extending vertically from the carrying plate in order to maintain the specimen collection tube in the upright position.

10. The diagnostic apparatus of claim 7, wherein the specimen collection tube handling compartment further comprises:
a tube disinfection means for disinfecting the specimen collection tube on the tube transportation means.

11. The diagnostic apparatus of claim 10, wherein the tube transportation means further comprises:
a horizontal rotation drive unit configured to transmit a rotational force in a horizontal direction to the tube accommodation unit; and
the tube disinfection means for disinfecting the specimen collection tube accommodated in the tube accommodation unit rotated horizontally by the horizontal rotation drive unit.

12. The diagnostic apparatus of claim 7, wherein the tube transportation means further comprises:
a tube discharge means for discharging in a discharge direction the specimen collection tube reaching a downstream side of the transportation unit.

13. The diagnostic apparatus of claim 12, wherein the tube discharge means comprises:
a discharging direction rotation drive unit for rotating in the discharge direction the tube accommodation unit reaching the downstream side of the transportation unit, without deviating from the transportation unit.

14. The diagnostic apparatus of claim 1, wherein the specimen obtainment means comprises:
an arm entry portion;
an arm body portion extending from the arm entry portion toward the specimen obtainment room, and
a cover portion configured to open and close the arm entry portion.

15. The diagnostic apparatus of claim 14, wherein the specimen obtainment means further comprises:
a pressure detection means for detecting pressure within the arm body portion in a state where the cover portion is closed.

16. The diagnostic apparatus of claim 15, wherein the pressure detection means comprises:
a through-hole for measuring pressure, the through-hole being formed in the cover portion;
an air hose being connected to the through-hole and extending to the outside; and
a pressure measurement means connected to the air hose.

17. The diagnostic apparatus of claim 14, wherein the specimen obtainment means further comprises:
a heater configured to heat the arm body portion.

18. The diagnostic apparatus of claim 1, further comprising:
an energy storage system or an uninterruptible power supply.

19. The diagnostic apparatus of claim 1, wherein the diagnostic apparatus is a mobile diagnostic apparatus.
